(19) United States
(12) Patent Application Publication
(10) Pub. No.: US 2026/0007354 A1
(43) Pub. Date: Jan. 8, 2026

(54) PERIPHERAL NERVE SIGNAL ACQUISITION METHOD AND SYSTEM

(71) Applicant: Fasikl Incorporated, DALLAS, TX (US)

(72) Inventor: Zhi Yang, DALLAS, TX (US)

(21) Appl. No.: 19/321,671

(22) Filed: Sep. 8, 2025

(30) Foreign Application Priority Data
Aug. 5, 2025 (CN) 202511087179.3

(51) Int. Cl. A61B 5/388; A61B 5/00; A61B 5/256; A61B 5/294; A61B 5/311

(52) U.S. Cl. CPC A61B 5/388; A61B 5/0051; A61B 5/256; A61B 5/294; A61B 5/311; A61B 5/6824; A61B 5/7203; A61B 5/7278; A61B 2560/0468

(57) ABSTRACT

The present disclosure provides a peripheral nerve signal acquisition method and system. The method includes: wearing a wearable device on a human wrist, disposing a perturbation unit in the wearable device, and applying mechanical perturbations perpendicular to a neural pathway to a skin surface during electrical stimulation cycles; under the action of the mechanical perturbations, acquiring a first signal, a second signal, and a third signal; performing multi-channel time-delay encoding on the first signal and the second signal to generate an interference signal set with temporal phase differences; constructing an artifact template signal associated with the electrical stimulation based on the third signal; performing artifact cancellation processing on the first signal and the second signal; and performing spatial analytical analysis in combination with a peripheral nerve pathway map, identifying an initiation site and a conduction pathway of peripheral nerve discharges, and outputting authentic discharge signals of the peripheral nerves.

### TECHNICAL FIELD

The present disclosure belongs to the field of physiological signal processing, and particularly relates to a peripheral nerve signal acquisition method and system.

### BACKGROUND

The peripheral nervous system is an important component for motion control and body sensing of external stimulation, and precise acquisition of electrophysiological signals is of great significance to neurological disease diagnosis, rehabilitation therapy, and neural interface research. Conventional neural signal acquisition methods mainly rely on invasive electrodes in direct contact with nerve tissues to obtain high-quality signals. Although the signal quality is superior, these methods are constrained by high surgical risks, poor biocompatibility, and limited long-term stability, thereby impeding applications in daily monitoring and long-term follow-ups.

To enhance the safety and use convenience, diverse non-invasive acquisition means have been developed in recent years, including surface electromyographic (EMG) electrodes, dry electrode arrays, and flexible adhesive electrodes. These methods, which typically acquire body surface potential variations induced by neural activities through conformal attachment to skin surfaces, have the advantages of wearing convenience and non-invasive safety, but also exhibit serious technical bottlenecks. First, neural signals exhibit a weak intensity which, when attenuated through multilayer tissue barriers, is susceptible to obscuration by noise. Second, artifact signals induced by electrical stimulation or muscular activities contaminate authentic neural signals, thereby resulting in identification difficulties. Third, body surface electrodes are incapable of precisely tracing neural discharge pathways, such that the signal localization capability is insufficient.

### SUMMARY

To solve the problems in the prior art, the present disclosure provides a peripheral nerve signal acquisition method, and the method includes the following steps:
wearing a wearable device on a human wrist, where the wearable device provides a multilayer electrode structure including a plurality of acquisition electrodes, and the multilayer electrode structure includes:
at least one set of surface electrodes, configured to acquire aggregated electrophysiological signals within a target region;
at least one set of piezoresponsive electrodes disposed beneath the surface electrodes, and configured to detect localized potential variations within a subcutaneous peripheral nerve region; and
at least one set of artifact detecting electrodes disposed orthogonally to a peripheral nerve pathway, and configured to acquire electrical stimulation artifact signals;
disposing a perturbation unit in the wearable device, where the perturbation unit is configured to apply mechanical perturbations perpendicular to the neural pathway to a skin surface during electrical stimulation cycles;
acquiring a first signal and a second signal respectively under the action of the mechanical perturbations, where the first signal represents an aggregated electrophysiological signal acquired by the surface electrodes, and the second signal represents a localized subcutaneous potential variation acquired by the piezoresponsive electrodes; and simultaneously acquiring a third signal through the artifact detecting electrodes;
performing multi-channel time-delay encoding on the first signal and the second signal to generate an interference signal set with temporal phase differences;
constructing an artifact template signal associated with electrical stimulation based on the third signal;
performing artifact cancellation processing on the first signal and the second signal based on the interference signal set and the artifact template signal to obtain artifact-suppressed enhanced neural signals; and
performing spatial analytical analysis in combination with a peripheral nerve pathway map based on the enhanced neural signals, identifying an initiation site and a conduction pathway of peripheral nerve discharges, and outputting authentic discharge signals of the peripheral nerves.

Further, the orthogonal direction refers to a direction forming a 90° angle with a longitudinal axis of a forearm.

Further, a difference between a frequency of the mechanical perturbations and a natural mechanical resonance frequency of local tissues is less than a preset value.

Further, excitation of the perturbation unit is phase-offset from the electrical stimulation signal by half a cycle.

Further, the first signal and the second signal are routed to a general-purpose amplifier via a dual-channel differential mode.

Further, the interference signal set is constructed as a two-dimensional matrix structure, and each element corresponds to a superimposed output of the first signal and the second signal under different delay combinations.

Further, the artifact template signal refers to a representative waveform curve generated from electrophysiological signals acquired by the artifact detecting electrodes across a plurality of stimulation cycles through temporal alignment, average superposition, and filtering processing.

Further, the enhanced neural signals are acquired by performing artifact template fitting on respective channel signals within the interference signal set and suppressing the fitted components.

The present disclosure further provides a peripheral nerve signal acquisition system, and the system includes the following modules:
a wearable device worn on a human wrist, where the wearable device is provided with a multilayer electrode structure including a plurality of acquisition electrodes, and the multilayer electrode structure includes:
at least one set of surface electrodes configured to acquire aggregated electrophysiological signals within a target region;
at least one set of piezoresponsive electrodes disposed beneath the surface electrodes, and configured to detect localized potential variations within a subcutaneous peripheral nerve region; and
at least one set of artifact detecting electrodes disposed orthogonally to a peripheral nerve pathway, and configured to acquire electrical stimulation artifact signals;
a perturbation unit disposed in the wearable device, where the perturbation unit is configured to apply mechanical perturbations perpendicular to the neural pathway to a skin surface during electrical stimulation cycles;
a signal acquisition module configured to acquire a first signal, a second signal, and a third signal under the action of the mechanical perturbations, where the first signal represents an aggregated electrophysiological signal acquired by the surface electrodes, and the second signal represents a localized subcutaneous potential variation acquired by the piezoresponsive electrodes, and the third signal represents an electrical stimulation artifact signal acquired by the artifact detecting electrodes;
an encoding module configured to perform multi-channel time-delay encoding on the first signal and the second signal to generate an interference signal set with temporal phase differences;
an artifact construction module configured to construct an artifact template signal associated with electrical stimulation based on the third signal;
an artifact cancellation module configured to perform an artifact cancellation operation based on the interference signal set and the artifact template signal to obtain artifact-suppressed enhanced neural signals; and
an analytical analysis module configured to perform spatial analytical analysis in combination with a peripheral nerve pathway map based on the enhanced neural signals, identify an initiation site and a conduction pathway of peripheral nerve discharges, and output authentic discharge signals of the peripheral nerves.

Further, the orthogonal direction refers to a direction forming a 90° angle with a longitudinal axis of a forearm.

Further, a difference between a frequency of the mechanical perturbations and a natural mechanical resonance frequency of local tissues is less than a preset value.

Further, excitation of the perturbation unit is phase-offset from the electrical stimulation signal by half a cycle.

Further, the first signal and the second signal are routed to a general-purpose amplifier via a dual-channel differential mode.

Further, the interference signal set is constructed as a two-dimensional matrix structure, and each element corresponds to a superimposed output of the first signal and the second signal under different delay combinations.

Further, the artifact template signal refers to a representative waveform curve generated from electrophysiological signals acquired by the artifact detecting electrodes across a plurality of stimulation cycles through temporal alignment, average superposition, and filtering processing.

Further, the enhanced neural signals are acquired by performing artifact template fitting on respective channel signals within the interference signal set and suppressing the fitted components.

In the present disclosure, amplification response of weak neural signals and effective cancellation of artifact components are achieved by constructing a multilayer structure having surface electrodes, piezoresponsive electrodes, and artifact detecting electrodes, and combining mechanical perturbations and time-delay interference mechanisms, such that the observability and spatiotemporal discriminability of neural signals are thereby effectively enhanced. Particularly for low-amplitude discharge events in a resting state, high-sensitivity acquisition is further achieved.

By incorporating the peripheral nerve pathway map and spatial analytical analysis, the initiation site of neural discharges is identified, the conduction pathway within the peripheral nerve structures is derived, and the signal source localization accuracy is significantly enhanced, which provides a solid foundation for peripheral nerve monitoring and electrophysiological modeling. This solution collectively offers the comprehensive advantages of non-invasiveness, high confidence, superior resolution, and strong robustness, and is suitable for practical deployment of wearable neural interface systems.

### BRIEF DESCRIPTION OF THE DRAWINGS

In order to explain the technical solutions in embodiments of the present disclosure or the prior art more clearly, the drawings to be used in the embodiments will be briefly introduced below. Obviously, the drawings described below are only some embodiments of the present disclosure. For those of ordinary skill in the art, other drawings may be obtained based on these drawings without making creative efforts.

FIG. 1 is a main flowchart of a method of the present disclosure.

FIG. 2 is a signal processing schematic diagram for artifact template construction and cancellation according to the present disclosure.

### DETAILED DESCRIPTIONS OF THE EMBODIMENTS

The present disclosure is described below in a preferred manner in combination with the drawings and the specific embodiments.

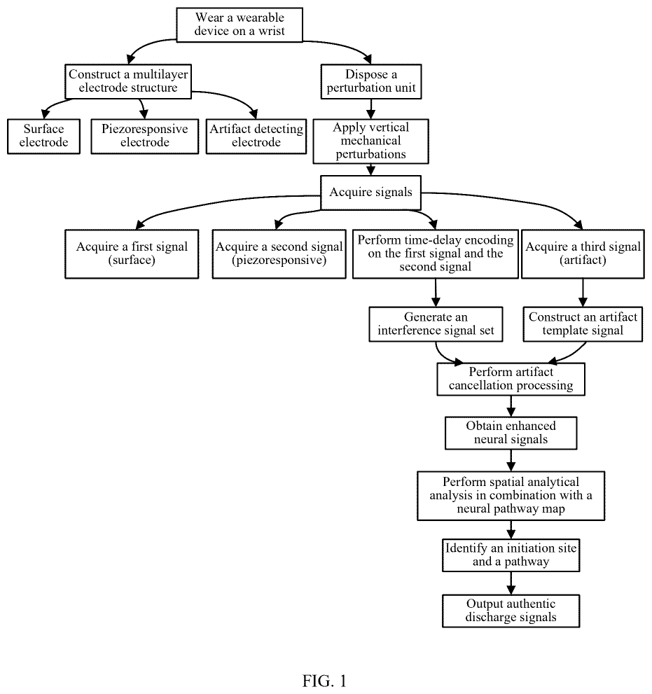

As shown in FIG. 1, in an embodiment, a peripheral nerve signal acquisition method according to the present disclosure, based on multi-source electrophysiological signal integration and adaptive artifact recognition mechanisms, is designed to non-invasively and sensitively extract stimulation or natural activity-evoked authentic discharge signals in a peripheral nervous system from a body surface, so as to provide high-quality input data for neural state assessment, neurological disease monitoring, and closed-loop neuromodulation.

The method specifically includes the following steps:
a wearable device is worn on a human wrist, where the wearable device provides a multilayer electrode structure including a plurality of acquisition electrodes, and the multilayer electrode structure includes:
at least one set of surface electrodes configured to acquire aggregated electrophysiological signals within a target region;
at least one set of piezoresponsive electrodes disposed beneath the surface electrodes, and configured to detect localized potential variations within a subcutaneous peripheral nerve region; and
at least one set of artifact detecting electrodes disposed orthogonally to a peripheral nerve pathway, and configured to acquire electrical stimulation artifact signals;
a perturbation unit is disposed in the wearable device, where the perturbation unit is configured to apply mechanical perturbations perpendicular to the neural pathway to a skin surface during electrical stimulation cycles;
a first signal and a second signal are acquired respectively under the action of the mechanical perturbations, where the first signal represents an aggregated electrophysiological signal acquired by the surface electrodes, and the second signal represents a localized subcutaneous potential variation acquired by the piezoresponsive electrodes; and simultaneously acquiring a third signal through the artifact detecting electrodes;
multi-channel time-delay encoding is performed on the first signal and the second signal to generate an interference signal set with temporal phase differences;
an artifact template signal associated with electrical stimulation is constructed based on the third signal;
artifact cancellation processing is performed on the first signal and the second signal based on the interference signal set and the artifact template signal to obtain artifact-suppressed enhanced neural signals; and
spatial analytical analysis is performed in combination with a peripheral nerve pathway map based on the enhanced neural signals, an initiation site and a conduction pathway of peripheral nerve discharges are identified, and authentic discharge signals of the peripheral nerves are outputted.

According to the peripheral nerve signal acquisition method, based on the non-invasive multilayer electrode structure and a mechanical perturbation triggering mechanism, synchronous sensing of multi-source bioelectrical signals from peripheral nerve regions is achieved by constructing a spatially distributed system including the surface electrodes, piezoresponsive electrodes, and artifact-detecting electrodes on the skin surface. Responsive potential perturbations are induced within peripheral nerve structures by applying directional mechanical stimulation to the skin through the perturbation unit, signals originating from superficial and subcutaneous layers are further decoupled and enhanced by time-delay encoding processing and phase interference algorithms, and signals are concurrently acquired by the artifact detecting electrodes to construct a stimulation artifact template, which achieves dynamic identification and adaptive suppression of non-neural interference signals. Ultimately, both the initiation site and the conduction pathway of peripheral nerve discharge sources are precisely reconstructed by a spatial analytical analysis method of the neural pathway map, and thus high-fidelity neural discharge data streams are outputted.

By constructing the multilayer non-invasive electrode structure and combining mechanical perturbation triggering and multi-channel time-delay interference encoding technologies, high-sensitivity acquisition of authentic subcutaneous peripheral nerve discharge signals is achieved, electrical stimulation-induced artifact interference is effectively suppressed, and a spatial resolution and a signal-to-noise ratio (SNR) of neural signals are significantly enhanced, such that high-precision and high-reliability neural electrophysiological input signals are provided for neural state assessment, disease monitoring, and closed-loop neuromodulation.

To further explain the above solution, the principles, embodiments, and technical effects of each step in the above solution will be described below in detail.

To achieve high-fidelity acquisition of authentic discharge activities in subcutaneous peripheral nerves under non-invasive conditions, the present disclosure provides a wearable device constructed on the human wrist. The device is of a multilayer heterogeneous electrode structure to achieve fine-grained separation and synergistic sensing of signals at different spatial depths and from different signal sources. This structure acquires composite electrophysiological signals through the surface electrodes, enhances a sensitivity to subcutaneous perturbation signals through the piezoresponsive electrodes, and achieves active sensing of stimulation signal artifacts through the artifact detecting electrodes. Therefore, spatiotemporal characteristic waveforms originating from authentic peripheral nerve activities are acquired without penetrating skin tissues.

In the present disclosure, during specific electrical stimulation cycles, a controlled electrical signal is applied to the skin surface to induce generation of detectable neural discharge events within a target peripheral nerve region. The electrical stimulation signal may be a low-frequency square wave, a capacitively coupled alternating current signal, or other biocompatible excitation current signals. The parameters of the electrical stimulation signal include: a stimulation frequency of 1 Hz to 1,000 Hz, a stimulation voltage of 1 V to 100 V, or a current intensity of 0.1 mA to 20 mA, and specifically depend on an excitation threshold of the target nerve region. The electrical stimulation serves not only for neural excitation but also as a structural foundation for artifact template construction, acquisition synchronization triggering, and signal enhancement.

The wearable device in the present disclosure refers to an intelligent electronic device with a flexible structure that conformally attaches to the skin surface of the wrist stably, and the carrier materials may include polyimide films or thermoplastic elastomers.

The multilayer electrode structure refers to an arrangement of at least three layers of electrode units with independent acquisition functions, integrated in the device, where interlayer electrical isolation and flexible structural continuity are maintained through insulating films, capacitive isolation materials, or conductive gels between adjacent layers. The operating principle of each electrode type is explained and described below in detail.

The surface electrode belongs to the first type of electrodes. The surface electrode includes a conductive electrode array conformally attached to the skin surface, and the operating principle relies on a body-surface potential sensing mechanism. When neural discharges generate weak voltage gradients on the skin surface through tissue media, the surface electrode may detect and acquire the electrophysiological signal. The surface electrode is typically a silver-silver chloride (Ag/AgCl) electrode, or alternatively, a flexible conductive layer formed from carbon nanotube films or silver nanowires. These materials offer advantages of low electrical resistance, favorable conformal attachment to the skin surface, and insensitivity to variations in electrode-skin contact impedance. This electrode structure is mainly configured to acquire aggregated electrophysiological signals containing mixed components of neural signals, electromyographic interference, and electrical stimulation artifacts, which serve as a main input source for subsequent signal decomposition processes.

The piezoresponsive electrode belongs to the second type of electrodes. The piezoresponsive electrode is disposed beneath the surface electrode and conformally attached to the skin surface. The operating principle relies on a piezoelectric effect, where a piezoelectric material generates a detectable voltage signal on the surface when subjected to mechanical stress or displacement excitation. In this solution, a subcutaneous nerve, whether electrically stimulated or naturally activated, induces localized tissue micro-deformations. These deformations propagate transcutaneously toward the surface and are detected by the piezoelectric material, such that potential response signals related to neural activities are generated. Typical piezoelectric materials include polyvinylidene fluoride (PVDF) or zinc oxide (ZnO) nanowire films. Such electrode enhances a detection sensitivity to electrophysiological variations induced by micro-displacements of neural structures through electro-conversion of mechanical perturbations, and exhibits a directional sensitivity and a filtering capability.

The artifact detecting electrode belongs to the third type of electrodes. The artifact detecting electrode is disposed orthogonally to a main pathway of the peripheral nerves, and is separately coupled to an artifact channel. The orthogonal direction refers to a spatial direction exhibiting a vertical relationship with the main pathway of the peripheral nerves.

When the wearable device is worn on the wrist, the main pathway of the peripheral nerves typically extends in a longitudinal axis of a forearm, arranged from proximal to distal regions. Therefore, the orthogonal direction is a transverse direction forming a 90° angle with the conduction direction of the peripheral nerves, which typically extends radially or circumferentially along the forearm. The purpose of disposing the artifact detecting electrodes orthogonally is to exploit the distinction in spatial propagation characteristics between electrical stimulation artifacts and neural discharges: artifact signals exhibit strong isotropic diffusion on the body surface, while authentic neural signals propagate predominantly axially along neural pathways. By disposing the artifact detecting electrodes orthogonally, the sensing capability for non-neural interference components is enhanced, spatially distributed artifact signals induced by electrical stimulation are effectively separated and extracted, and independent channels and reference criteria are provided for subsequent artifact template construction and signal cancellation.

The artifact detecting electrode utilizes the directional spatial distribution of systematic electrical field interference induced by electrical stimulation, which exhibits a marked distinction from the conduction pathways of neural signals. This electrode prioritizes acquisition of non-neural noise signals associated with electrical stimulation, and particularly potential transition waveforms and field reflection signals induced by power excitation pulses, which serve as foundational inputs for subsequent artifact template generation. The electrode, made of high-resistance carbon-based materials, enhances responsiveness to high-frequency artifact components.

The surface electrode arrays are arranged on a flexible stretchable substrate to form a two-dimensional grid conformally attached to the wrist skin. The piezoresponsive electrodes are disposed beneath the surface electrodes via a printed lamination process, with alignment to corresponding regions of the surface electrodes to ensure three-dimensional signal decomposition within an identical spatial acquisition window. The artifact detecting electrodes are distributed at the peripheral zones of the device, and maintain physical isolation from primary signal channels. The electrode layers are isolated via insulating polymers, and structural compliance and signal conductivity are maintained through a flexible conductive adhesive layer.

In a preferred embodiment, the piezoresponsive electrode has a thickness controlled within the range of 20 to 30 μm and an interelectrode spacing of less than 3 mm. Additionally, the piezoresponsive electrode is integrated with a miniature amplification circuit module with a gain range of 10 to 100 times, configured to effectively amplify low-frequency weak neural displacement response signals. In another preferred embodiment, the artifact detecting electrode is coupled to a high-speed sampling channel with a sampling frequency exceeding 5 kHz, configured to capture high-frequency components within artifact signals and construct differential cancellation templates.

Structured enhancement of neural signal acquisition is achieved by performing spatial separation and functional decoupling of electrical stimulation artifacts from neural signals at a physical level, and synergistically constructing a high-SNR sensing mechanism using heterogeneous sensitivities of different electrode layers. This solution achieves neural signal localization and interference suppression without requiring any invasive operation, significantly enhances the signal acquisition quality, and is suitable for diverse application scenarios such as closed-loop neuromodulation systems, neuropathological status monitoring systems, and wearable neural interface platforms.

In a specific example, a wearable device worn on a right wrist includes 36 surface electrodes, 36 piezoresponsive electrodes, and 4 artifact detecting electrodes. The electrodes are coupled to a data processing chip via a multi-channel parallel acquisition module, and signal variations are synchronously recorded within electrical stimulation cycles based on a timing control circuit.

Electrophysiological activities induced by peripheral nerve discharges mainly exist in a form of local field potentials within biological organisms, a spatial conduction range of which is subject to shielding and attenuation effects imposed by tissues including skins, muscles, and fascias. Authentic neural discharges typically exhibit microvolt-level potential variations at the skin surface, and are highly susceptible to being obscured by noise or artifact signals. To enhance the response sensitivity of the non-invasive electrode system to subcutaneous peripheral nerve discharge activities, a perturbation unit is disposed in the wearable device, and is configured to actively apply mechanical perturbations to the skin surface during electrical stimulation cycles. Through such controlled external mechanical intervention, subtle deformations are induced in subcutaneous tissues, and neural activity-induced responses that are difficult to sense directly at the skin surface are amplified. Particularly under resting-state conditions or during low-amplitude discharges, localized potential fluctuations induced by neural activities are extremely weak and incapable of traversing multilayer biological tissues to reach the epidermis. By applying directional perturbations, tissue-coupled displacement is induced at a physical level, and thus the piezoresponsive electrodes are activated to generate observable electrical signals, which achieves indirect enhancement of neural discharge signals.

Specifically, such perturbation is defined as a low-intensity, frequency-controllable directional micro-vibration or transient impulse, which induces micro-scale relative displacement between the skin and underlying tissues during application. This perturbation mechanism leads to the following three electrophysiological effects:

Certain piezoresponsive materials, such as PVDF, undergo charge separation after bearing mechanical stress, thereby forming a potential difference. By subcutaneously disposing the piezoresponsive electrodes, perturbations induce micro-deformations within the tissue and cause the piezoelectric material to generate detectable responsive electrical signals, such that mechanical resonance signals triggered by neural discharges are indirectly captured.

The electrical impedance characteristics of biological tissues are influenced by a tissue density, a water content, and structural alignment. When mechanical perturbations lead to transient tissue compression or stretching, the electrical conductivity and dielectric constant vary, and thus the conduction pathways of neural signals are dynamically modulated, which is favorable for enhancing signal coherence and temporal focusing.

When the perturbation frequency approaches a natural mechanical resonant frequency of local tissues, structural resonance effects are excited at a microscopic level, and originally low-amplitude neurally evoked signals are amplified through a resonance coupling mechanism within specific frequency bands, which enhances a sensing sensitivity to such signals.

Accordingly, the perturbation is not a random perturbation, and incorporates defined directional and periodic parameters. The function is analogous to that of a “mechanical amplifier” or “tissue exciter” for neural responses, thereby not only enhancing electrophysiological signal accessibility but also avoiding interference or damage possibly caused by high-intensity stimulation.

Therefore, the perturbation unit of the present disclosure is a miniature mechanical actuator integrated into the wearable device, which exhibits a periodic and directional mechanical motion capability, and is configured to apply controlled perturbations to the skin surface in a specific direction.

In a specific implementation process, the perturbation unit may include the following optional structural forms:
a micro-oscillating plate driven by piezoelectric ceramics, an electromagnetically actuated micro-coil oscillator, or a periodic expansion mechanism made from thermally responsive materials.

The perturbation unit is disposed at a central or peripheral position of the electrode array and configured to operate synchronously with electrical stimulation through a controller. During electrical stimulation cycles, the perturbation unit is driven to operate at a set amplitude and frequency, perturbations are applied to the skin with an acceleration range of 0.1 to 2 mN/mm², and effective excitation of subcutaneous nerve tissues is achieved without inducing tissue discomfort.

Preferably, the perturbation unit employs a piezoelectric vibration source as a driving mechanism; and a driving voltage ranges from 3 V to 12 V, and an excitation frequency is adjustable, ranging from 20 Hz to 300 Hz. The vibration element is encapsulated in a central region of the multilayer electrode structure through a flexible encapsulation process, which ensures the directional consistency of mechanical perturbations without affecting electrode conformality.

Further preferably, the excitation mode of the perturbation unit is phase-offset from the electrical stimulation signal by half a cycle, thereby preventing frequency overlap between perturbation signals and artifact signals, and enhancing the independence and reliability of subsequent neural signal extraction.

By applying subtle mechanical perturbations to the skin surface in a specific direction, tissue micro-deformations are induced, such that a sensitivity of the piezoresponsive electrodes to neurally evoked electrophysiological signals is enhanced, and a high-precision detection of neural discharges is achieved under low-activation conditions. This approach, dispensing with skin penetration or high-voltage stimulation, enhances the sensing capability of the electrode system only through physical perturbations, which provides a high-SNR and high directional sensing mechanism for non-invasive neural acquisition devices.

In a specific example, in the above wearable device configured to be worn on the right wrist, the perturbation unit adopts a PVDF piezoelectric oscillator with a thickness of 0.6 mm, which is disposed centrally beneath the electrode array, and exhibits an operating frequency of 120 Hz and an operating voltage of 5 V. Upon emission of each electrical stimulation signal, the perturbation unit initiates after a delay of 4 ms and maintains operating for 8 ms. The structure, upon testing, maintains wearing comfort while inducing the piezoresponsive electrodes to generate an additional response of approximately 200 μV, such that a detectability of low-amplitude neural discharges is effectively enhanced.

To effectively capture subcutaneous peripheral nerve discharge activities and synchronously identify artifact interference signals, a multi-channel heterogeneous electrode structure is employed under the action of mechanical perturbations to separately acquire three signal types designated for surface signal sensing, deep-layer response extraction, and interference signal modeling.

Specifically, during application of directional mechanical perturbations, electrophysiological signals of different sources and action mechanisms are respectively acquired through the surface electrodes, piezoresponsive electrodes, and artifact detecting electrodes, thereby achieving spatial decoupling and synergistic extraction of electrophysiological signals, and further providing a raw data foundation for subsequent processing steps including neural signal enhancement, artifact cancellation, and discharge pathway identification.

The first signal refers to an aggregated electrophysiological signal acquired by the surface electrodes from the skin surface layer during perturbations, and includes neural signals, EMG interference, electrical stimulation responses, and other background physiological noise, thereby serving as the broadest primary signal source.

The second signal refers to a potential variation signal generated by the piezoresponsive electrodes in response to subcutaneous tissue deformations during the perturbation process, and reflects an electromechanical resonance effect induced by deep-layer neural activation.

The third signal refers to a non-neural interference signal directly correlated with electrical stimulation and acquired by the artifact detecting electrodes, and is typically manifested as a high-amplitude, short-duration, and frequency-concentrated pulse signal, and configured for subsequent artifact template construction.

Under mechanical perturbations triggered during electrical stimulation cycles, three types of electrodes are concurrently activated to perform synchronous signal acquisition. The surface electrodes preferably have a sampling frequency ranging from 1,000 Hz to 2,000 Hz, and are configured for high-temporal-resolution tracing of neural discharge processes. The piezoresponsive electrodes sense strain potentials generated by perturbations through a PVDF or ZnO film, and are coupled to a low-noise charge amplification module to enhance a voltage response sensitivity. The artifact detecting electrodes are disposed orthogonally to the neural pathway, and sampling channels preferably adopt a high-speed analog-to-digital conversion module operating at above 5 KHz to precisely characterize transient features of electrical stimulation interference signals. The temporal synchronization of the above three types of signals is implemented by a central control unit, and the acquisition process is strictly controlled within electrical stimulation and perturbation cycles to ensure that signal sources are traceable and response components are distinguishable.

In a preferred embodiment, the first signal and the second signal are routed to a general-purpose amplifier via a dual-channel differential mode to enhance a common-mode rejection ratio and improve a discriminability of subtle neural signals; additionally, the third signal is acquired and immediately routed to a local computing module to perform real-time artifact modeling operations, where the computing module outputs an artifact estimation value based on a delay-matched filtering architecture, which is invoked in subsequent signal cancellation steps.

Under unified stimulation-perturbation temporal control, multi-source heterogeneous electrophysiological signals are respectively acquired to accurately obtain target neural discharge information, simultaneously monitor perturbation responses and interference signals, and provide a spatiotemporally decoupled data foundation for subsequent signal enhancement and artifact cancellation, thereby significantly elevating the overall SNR and neural source localization capability of the system.

To achieve high-sensitivity extraction and interference suppression of authentic neural discharge signals, enhancement processing is performed on the first signal and the second signal by constructing an interference signal set. The interference signal set is configured to combine the first signal and the second signal under different time delays through a multi-channel time-delay encoding structure to form an interference output with phase difference characteristics, which aims to enhance the temporal consistency of authentic neural signals while suppressing the spatial diffusivity and temporal randomness of artifact interference.

Authentic peripheral nerve discharge signals, which typically originate from a fixed temporal point and propagate in a specific direction, exhibit temporal coherence in a multi-channel delay system. When the first signal and the second signal are combined after being delayed respectively and both originate from the same neural discharge event, the first signal and the second signal undergo constructive superposition under specific delay combinations, such that a peak response appears. Artifact signals, due to instantaneous global distribution characteristics and lack of temporal concentration, exhibit phase inconsistency in different channels, and thus are averaged or canceled upon superposition.

The interference signal set is constructed as a two-dimensional matrix structure, denoted as H_{i,j}(t); in the formula, i represents a delay channel index of the first signal, j represents a delay channel index of the second signal, and/represents a sampling time point. In each channel combination, the output signal H_{i,j}(t) is calculated by the following formula:

H_{i,j}(t)=S₁(t−dᵢ)+S₂(t−dⱼ); in the formula, S₁(t) represents the first signal, S₂(t) represents the second signal, dᵢ represents a temporal offset of the i^{th} delay channel, dᵢ represents a temporal offset of the j^{th} delay channel. All H_{i,j}(t) signals constitute the interference signal set for subsequent artifact cancellation and authentic neural signal extraction.

During specific implementation, the first signal and the second signal are respectively inputted into a plurality of delay channels, which may be implemented by first-in-first-out (FIFO) buffers, digital shift registers, or software delay modules in a field-programmable gate array (FPGA). Taking an FPGA implementation as an example, sampled signals S₁(t) and S₂(t) are respectively written into two independent FIFO buffer units, and each channel achieves a temporal offset by configuring reading delay differences dᵢ and dⱼ. In a main control logic, S₁(t) and S₂(t) are read out successively per channel and combined at corresponding delay timings to generate H_{i,j}(t) interference outputs. A number of channels is flexibly expandable; for example, each signal is configured with four delay channels, dᵢ takes 0, 5, 10, and 15, and dⱼ is similar, thereby forming 16 interference outputs.

After the interference signal set is formed, a relationship between a temporal dimension t and a channel delay combination index (i,j) may be analyzed in a three-dimensional tensor structure. Authentic neural signals generated at a time point t=t₀ are manifested as synchronous peaks in a plurality of H_{i,j}(t₀) signals, with positions (i₀,j₀) satisfying S₁(t) and S₂(t) coherent delay matching conditions. By determining the position of the maximum response in H_{i,j}(t), a temporal occurrence of neural events is localized, and a conduction difference between S₁(t) and S₂(t) is derived, thereby characterizing discharge pathway information.

The interference signal set has the following functions:

The amplitude response of authentic neural signals is enhanced, and such signals exhibit consistent peaks in a plurality of channels, which facilitates extraction of neural signals under low SNR conditions.

Electrical stimulation artifacts or other interference signals are suppressed; these signals exhibit inconsistent distribution in different channels and consequently fail to form a concentrated peak when superimposed.

Delay-channel joint features are provided for constructing neural conduction models and discharge initiation point tracking algorithms.

In a preferred embodiment, the interference signal set is configured not only for neural signal enhancement, but also configured as an input tensor to a neural recognition module; and a neural network model is further configured to extract delay distribution features to achieve classification of active states of different nerve bundles and spatial localization.

In a specific example, signals S1 and S2 acquired by the device are fed into four delay channels with respective delays of 0 ms, 5 ms, 10 ms, and 15 ms, thereby forming 16 interference channels. The system detects a maximum output amplitude at H23t at an 18-ms time point after stimulation, with a channel 2 corresponding to a 5-ms delay of S1 and a channel 3 corresponding to a 10-ms delay of S2. The corresponding phase-matching value at this position is consistent with the expected nerve conduction velocity, which further verifies an efficacy of the interference structure in authentic neural signal enhancement and temporal identification.

During peripheral nerve signal acquisition, electrical stimulation processes are invariably accompanied by numerous non-neural artifact signals arising primarily from mechanisms including surface current diffusion, skin polarization, and electrode capacitive coupling. Such artifact signals, characterized by a high amplitude, a prolonged duration, and a spatial diffusivity, tend to obscure authentic neural signals, thereby inducing misidentification or missed detection. To achieve effective separation and suppression of artifacts, the artifact detecting electrodes are utilized to acquire artifact signals, and an artifact template signal is constructed based on these signals, which provides a reference for subsequent signal cancellation algorithms.

The artifact template signal refers to a representative waveform curve generated from the electrophysiological signals acquired by the artifact detecting electrodes across a plurality of stimulation cycles through temporal alignment, average superposition, and filtering processing. This template is configured to reflect the typical temporal morphology and spatial amplitude features of artifacts under stimulation conditions, and serves as a structural reference for non-neural components.

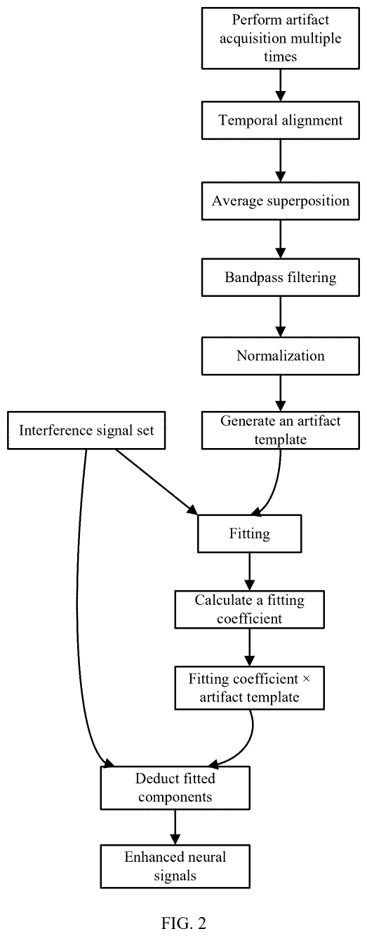

An artifact signal suppression process is illustrated in FIG. 2. Specifically, the temporal signal acquired by the artifact detecting electrodes after each stimulation is denoted as V⁽ⁱ⁾(t); in the formula, i represents a i^{th} sampling, t represents a sampling time point, and a sampling point count is denoted as N. After n acquisition iterations, an artifact template signal Mₜₚₗ(t) is constructed, and calculated with the following formula:

$${M}_{tpl} ( t ) = \frac{1}{n} {\sum }_{j = 1}^{n} {V}^{( i )} ( t ) ;$$

in the formula:
Mₜₚₗ(t) represents an artifact template signal constructed;
n represents a number of sampling times; and
V⁽ⁱ⁾(t) represents an artifact signal acquired at the i^{th} sampling.

Upon completion of template construction, bandpass filtering with a typical passband ranging from 20 Hz to 500 Hz is performed to suppress low-frequency drift and high-frequency noise. Subsequently, normalization is performed to constrain the maximum amplitude to 1, which facilitates subsequent matching and amplitude fitting.

In a preferred embodiment, an exponential weighted moving average update mechanism is employed to perform online iteration of the template, so as to enhance an adaptability of the template to long-term stimulation environmental variations. An update formula is as follows:

$${M}_{tpl}^{new} ( t ) = α \cdot {V}^{new} ( t ) + ( 1 - α ) \cdot {M}_{tol}^{old} ( t ) ;$$

in the formula:

$${M}_{tpl}^{new} ( t )$$

represents an updated artifact template;
V^{new}(t) represents an artifact signal acquired in the current stimulation cycle;

$${M}_{tpl}^{old} ( t )$$

represents an artifact template derived from the preceding sampling; and
α represents an update weight parameter, ranging from 0 to 1.

Through this mechanism, minor adjustment may be performed on the template based on the most recent artifact waveform, such that the template progressively adapts to dynamic changes in stimulation amplitude, stimulation frequency, and electrode states, and a fitting degree of the template to real-world scenarios is enhanced.

The constructed artifact template based on the above approach stably reflects the spatiotemporal features of electrical stimulation artifacts, and high-confidence structural fitting is achieved during subsequent artifact cancellation. Consequently, erroneous suppression of authentic neural signals is effectively prevented due to similar morphology, and the overall temporal precision and stability of the system for signal processing are simultaneously enhanced.

Continuing with the above embodiment of the wrist-worn device, the system is configured to record artifact signals within 50 ms after each stimulation, with a sampling frequency of 1,000 Hz and a sampling point count N of 50. Following continuous recording over n=20 iterations, Mₜₚₗ(t) is constructed by averaging. The detection results reveal that the template signal exhibits a peak at approximately 12 ms, with a corresponding average amplitude of 90 μV and a standard deviation of 4 μV.

In peripheral nerve signal acquisition, electrical stimulation artifact signals, characterized by high amplitudes, prolonged durations, and stereotyped recurrence, frequently severely interfere with identification and analysis of authentic neural discharge signals. Owing to the significant disparity between the spatial diffusivity of artifact signals and the localized nature of authentic neural signals, combined utilization of the interference signal set and the artifact template signal achieves phase identification, amplitude fitting, and structural cancellation of artifacts. Accordingly, authentic neural signal components are retained, and artifact-suppressed enhanced neural signals are obtained.

Artifact cancellation processing refers to fitting and subtraction from the original signals of potential artifact components in each interference channel by performing amplitude matching and structural matching using an artifact template signal in the interference signal set constituted by multi-channel time-delay encoding. This processing approach is based on two assumptions: first, the artifact signals exhibit stable and predictable structural features in various channels; and second, authentic neural signals demonstrate coherent phase difference features in different delay paths.

In a specific implementation process, the interference signal set is defined as H_{i,j}(t), and the artifact template signal is defined as Mₜₚₗ(t). The interference output of each channel may then be expressed as follows:

$${H}_{i , j} ( t ) = {R}_{i , j} ( t ) + {A}_{j} \cdot {M}_{tpl} ( t ) ;$$

in the formula,
R_{i,j}(t) represents an authentic neural signal component in the corresponding channel; and
Aⱼ represents an artifact amplitude fitting coefficient of that channel.
Aⱼ is determined through a least mean square error fitting algorithm to minimize the error in the following formula:

$$min {\sum }_{t} {( {H}_{i , j} ( t ) - {A}_{j} \cdot {M}_{tpl} ( t ) )}^{2} .$$

Subsequently, the fitted artifact component is deducted from the interference signal:

$${R}_{i , j} ( t ) = {H}_{i , j} ( t ) - {A}_{j} \cdot {M}_{tpl} ( t ) ;$$

R_{i,j}(t) represents an artifact-suppressed enhanced neural signal.

In a preferred embodiment, a weighted matching mechanism is incorporated; an artifact weighting function w(t) is defined, and this function is assigned with elevated weights during peak artifact periods to enhance a matching precision. The updated fitting coefficient Aⱼ is calculated as follows:

$${A}_{j} = \frac{{\sum }_{t} {H}_{i , j} ( t ) \cdot {M}_{tpl} ( t ) \cdot w ( t )}{{\sum }_{t} {{M}_{tpl} ( t )}^{2} \cdot w ( t )} .$$

Additionally, regularization may be employed to constrain a range of Aⱼ, thereby preventing signal distortion induced by overfitting.

The artifact cancellation processing implemented by the above approach enables precise identification and modeling of artifact waveforms in a multi-channel redundant architecture to effectively separate neural signals from interference components; enhances the observability of weak neural signals, and improves the signal quality and recognition confidence; and exhibits favorable temporal continuity and real-time processing capability, and is suitable for continuous signal decomposition under high-frequency stimulation conditions.

Continuing with the above embodiment, upon completion of the interference signal set construction, responses exhibiting a high degree of matching with the artifact template are detected in a plurality of channels at a 12-ms position. The system calculates the Aⱼ value of the corresponding channel to range from 0.8 to 1.1. Following the above cancellation processing, the wide-peak signal originally present at 12 ms in the interference channel output is substantially canceled, and subsequently, temporally aligned narrow-peak signals emerge in a plurality of channels between 15 ms and 18 ms. The system identifies these signals as authentic peripheral nerve discharge signals, and finally extracts the initiation site and the conduction pathway of these signals successfully.

During peripheral nerve signal acquisition, although artifact interference has been attenuated in the enhanced neural signals, source localization ambiguity and signal aliasing problems persist due to the spatial distribution features of signals acquired by the surface electrodes. Peripheral nerve signals exhibit defined spatiotemporal conduction patterns, manifested as propagating from a discharge initiation site to a terminal region along specific neural pathways at a specific velocity. Based on this physiological characteristic, spatial analytical analysis combined with a predetermined peripheral nerve pathway map enables precise identification of the neural discharge initiation site and conduction pathway, thereby further reconstructing authentic neural discharge signals.

The peripheral nerve pathway map serves as a model structure delineating the spatial distribution structure and conduction property of peripheral nerve bundles. This map, as a spatial structural prior for neural signal analytical analysis, provides key information regarding nerve discharge conduction pathways, directionality, and geometric distances to support signal source localization and pathway tracing.

The establishment approaches of the map include two options. One option is a prior modeling approach based on anatomical structures, where anatomical parameter models for common neural pathways in specific regions such as wrists, elbows, and shoulder are constructed using human anatomical literature, medical images, or EMG and neurographic data. The neural trunk is stored as a three-dimensional spatial pathway, and each pathway is constituted by a sequence of continuous coordinate points, and accompanied by the average conduction velocity per pathway segment, the pathway length, and the nerve name. The other option is a self-learning modeling approach based on neural signal data, where spatiotemporal correlations between electrodes are modeled using graph optimization algorithms or graph neural networks by acquiring a plurality of neural electrical activity signal sets under different motion or stimulation conditions, and a graphic structure that fits actual neural conduction directions is generated. Optional pathway generation methods include a pathway alignment method based on dynamic time warping, a shortest conduction pathway extraction method in graph structures, or a functional neural cluster automatic division method based on cluster analysis.

The neural pathway map is ultimately represented as a directed graphic structure G, where a node set N represents respective electrodes or anatomical key points, and an edge set E represents neural pathway segments, each edge contains the following fields: a three-dimensional spatial vector represents spatial position difference from node i to node j; a conduction velocity represents an average signal conduction velocity over the pathway segment; and a functional designation represents whether the pathway is a trunk, a branch, or a putative pathway.

The spatial analytical analysis refers to a process in which enhanced neural signals acquired from a plurality of electrode channels at different positions are mathematically mapped onto the neural pathway map to derive the signal source site and conduction process.

In a specific implementation process, the enhanced neural signal is defined as Eᵢ(t), representing a signal value at the i^{th} electrode at time t; the neural pathway map is defined to include M candidate pathways, each pathway is defined as Pₘ, a conduction velocity is defined as vₘ, and a distance from an electrode to an initiation site of the pathway is defined as d_{i,m}. Theoretically, when a pathway is an authentic neural signal conduction pathway, signals on different electrodes satisfy the following delay relationship:

$${τ}_{i , m} = \frac{{d}_{i , m}}{{v}_{m}} .$$

Based on this relationship, an inverse analytical model is constructed to perform matching calculations on each pathway, and a matching index Dₘ is defined as follows:

$${D}_{m} = {\sum }_{i} {E}_{i} ( {t}_{0} + {τ}_{i , m} ) ;$$

in the formula, t₀ represents an assumed starting time point.

The maximum Dₘ is selected from all pathways, and the corresponding pathway Pₘ is identified as an optimal conduction pathway for neural discharges.

In a preferred embodiment, a multi-parameter optimization algorithm based on gradient descent or maximum likelihood estimation is utilized to search for an optimal pathway and initiation site combination in a pathway set. Further, a graph neural network structure may be incorporated to perform fine-grained modeling and dynamic updating of the neural pathway map by training historical signal samples, such that the identification accuracy rate is enhanced.

The technical effects of the above method are as follows: spatial structural features of peripheral nerve signals are fully utilized to enhance a signal inversion accuracy, achieve accurate determination of the signal source site and conduction direction, solve signal aliasing problems caused by limited spatial electrode distribution, and enhance the comprehensive capability of the system to sense spatiotemporal behavior of neural events.

Continuing with the above embodiment, in the electrode array worn on the right wrist, enhanced neural signals E₁(t) to E₈(t) acquired are subjected to matching calculations against the map model including ulnar, median, and radial nerves through the spatial analytical analysis module; it is determined that signal delays of electrodes along a pathway P3 exhibit an increasing trend and a maximum phase correlation; and the neural discharge is finally determined to originate from a distal region of the wrist corresponding to the electrode 2, and propagate proximally along the ulnar nerve pathway at approximately 45 m/s. This discharge pattern is thereby identified as an authentic neural discharge signal.

In another embodiment, the present disclosure further provides a peripheral nerve signal acquisition system, and the system includes:
a wearable device worn on a human wrist, where the wearable device is provided with a multilayer electrode structure including a plurality of acquisition electrodes, and the multilayer electrode structure includes:
at least one set of surface electrodes configured to acquire aggregated electrophysiological signals within a target region;
at least one set of piezoresponsive electrodes disposed beneath the surface electrodes, and configured to detect localized potential variations within a subcutaneous peripheral nerve region; and
at least one set of artifact detecting electrodes disposed orthogonally to a peripheral nerve pathway, and configured to acquire electrical stimulation artifact signals;
a perturbation unit disposed in the wearable device, where the perturbation unit is configured to apply mechanical perturbations perpendicular to the neural pathway to a skin surface during electrical stimulation cycles;
a signal acquisition module configured to acquire a first signal, a second signal, and a third signal under the action of the mechanical perturbations, where the first signal represents an aggregated electrophysiological signal acquired by the surface electrodes, and the second signal represents a localized subcutaneous potential variation acquired by the piezoresponsive electrodes, and the third signal represents an electrical stimulation artifact signal acquired by the artifact detecting electrodes;
an encoding module configured to perform multi-channel time-delay encoding on the first signal and the second signal to generate an interference signal set with temporal phase differences;
an artifact construction module configured to construct an artifact template signal associated with electrical stimulation based on the third signal;
an artifact cancellation module configured to perform an artifact cancellation operation based on the interference signal set and the artifact template signal to obtain artifact-suppressed enhanced neural signals; and
an analytical analysis module configured to perform spatial analytical analysis in combination with a peripheral nerve pathway map based on the enhanced neural signals, identify an initiation site and a conduction pathway of peripheral nerve discharges, and output authentic discharge signals of the peripheral nerves.

In a further embodiment, the orthogonal direction refers to a direction forming a 90° angle with a longitudinal axis of a forearm.

In a further embodiment, a difference between a frequency of the mechanical perturbations and a natural mechanical resonance frequency of local tissues is less than a preset value.

In a further embodiment, excitation of the perturbation unit is phase-offset from the electrical stimulation signal by half a cycle.

In a further embodiment, the first signal and the second signal are routed to a general-purpose amplifier via a dual-channel differential mode.

In a further embodiment, the interference signal set constitutes a two-dimensional matrix structure, and each element corresponds to a superimposed output of the first signal and the second signal under different delay combinations.

In a further embodiment, the artifact template signal refers to a representative waveform curve generated from the electrophysiological signals acquired by the artifact detecting electrodes across a plurality of stimulation cycles through temporal alignment, average superposition, and filtering processing.

In a further embodiment, the enhanced neural signals are acquired by performing artifact template fitting on respective channel signals within the interference signal set and suppressing the fitted components.

It should be noted that the above explanation of the embodiments of the peripheral nerve signal acquisition method is further applicable to devices in the embodiments of the present disclosure, which will not be repeated herein.

The foregoing descriptions only represent specific embodiments of the present disclosure. Any alterations or substitutions readily conceivable by those skilled in the art within the technical scope disclosed herein should be construed as falling within the protection scope of the present disclosure. The module structures not explicitly defined in the present disclosure are subject to the contents recorded in the prior art. The prior art mentioned in the Background and Detailed Description sections of the present disclosure may be incorporated herein to understand the meanings of certain technical features or parameters.

What is claimed is:

1. A peripheral nerve signal acquisition method, wherein the method comprises the following steps:
wearing a wearable device on a human wrist, wherein the wearable device provides a multilayer electrode structure comprising a plurality of acquisition electrodes, and the multilayer electrode structure comprises:
at least one set of surface electrodes configured to acquire aggregated electrophysiological signals within a target region;
at least one set of piezoresponsive electrodes disposed beneath the surface electrodes, and configured to detect localized potential variations within a subcutaneous peripheral nerve region; and
at least one set of artifact detecting electrodes disposed orthogonally to a peripheral nerve pathway, and configured to acquire electrical stimulation artifact signals;
disposing a perturbation unit in the wearable device, wherein the perturbation unit is configured to apply mechanical perturbations perpendicular to the neural pathway to a skin surface during electrical stimulation cycles;
acquiring a first signal and a second signal respectively under the action of the mechanical perturbations, wherein the first signal represents an aggregated electrophysiological signal acquired by the surface electrodes, and the second signal represents a localized subcutaneous potential variation acquired by the piezoresponsive electrodes; and simultaneously acquiring a third signal through the artifact detecting electrodes;
performing multi-channel time-delay encoding on the first signal and the second signal to generate an interference signal set with temporal phase differences;
constructing an artifact template signal associated with electrical stimulation based on the third signal;
performing artifact cancellation processing on the first signal and the second signal based on the interference signal set and the artifact template signal to obtain artifact-suppressed enhanced neural signals; and
performing spatial analytical analysis in combination with a peripheral nerve pathway map based on the enhanced neural signals, identifying an initiation site and a conduction pathway of peripheral nerve discharges, and outputting authentic discharge signals of the peripheral nerves.

2. The peripheral nerve signal acquisition method according to claim 1, wherein the orthogonal direction refers to a direction of forming a 90° angle with a longitudinal axis of a forearm.

3. The peripheral nerve signal acquisition method according to claim 1, wherein a difference between a frequency of the mechanical perturbations and a natural mechanical resonance frequency of local tissues is less than a preset value.

4. The peripheral nerve signal acquisition method according to claim 1, wherein excitation of the perturbation unit is phase-offset from the electrical stimulation signal by half a cycle.

5. The peripheral nerve signal acquisition method according to claim 1, wherein the first signal and the second signal are routed to a general-purpose amplifier via a dual-channel differential mode.

6. The peripheral nerve signal acquisition method according to claim 1, wherein the interference signal set constitutes a two-dimensional matrix structure, and each element corresponds to a superimposed output of the first signal and the second signal under different delay combinations.

7. The peripheral nerve signal acquisition method according to claim 1, wherein the artifact template signal refers to a representative waveform curve generated from the electrophysiological signals acquired by the artifact detecting electrodes across a plurality of stimulation cycles through temporal alignment, average superposition, and filtering processing.

8. The peripheral nerve signal acquisition method according to claim 1, wherein the enhanced neural signals are acquired by performing artifact template fitting on respective channel signals within the interference signal set and suppressing the fitted components.

9. A peripheral nerve signal acquisition system, wherein the system comprises:
a wearable device worn on a human wrist, wherein the wearable device is provided with a multilayer electrode structure comprising a plurality of acquisition electrodes, and the multilayer electrode structure comprises:
at least one set of surface electrodes configured to acquire aggregated electrophysiological signals within a target region;
at least one set of piezoresponsive electrodes disposed beneath the surface electrodes, and configured to detect localized potential variations within a subcutaneous peripheral nerve region; and
at least one set of artifact detecting electrodes disposed orthogonally to a peripheral nerve pathway, and configured to acquire electrical stimulation artifact signals;
a perturbation unit disposed in the wearable device, wherein the perturbation unit is configured to apply mechanical perturbations perpendicular to the neural pathway to a skin surface during electrical stimulation cycles;
a signal acquisition module configured to acquire a first signal, a second signal, and a third signal under the action of the mechanical perturbations, wherein the first signal represents an aggregated electrophysiological signal acquired by the surface electrodes, and the second signal represents a localized subcutaneous potential variation acquired by the piezoresponsive electrodes, and the third signal represents an electrical stimulation artifact signal acquired by the artifact detecting electrodes;
an encoding module configured to perform multi-channel time-delay encoding on the first signal and the second signal to generate an interference signal set with temporal phase differences;
an artifact construction module configured to construct an artifact template signal associated with electrical stimulation based on the third signal;
an artifact cancellation module configured to perform an artifact cancellation operation based on the interference signal set and the artifact template signal to obtain artifact-suppressed enhanced neural signals; and
an analytical analysis module configured to perform spatial analytical analysis in combination with a peripheral nerve pathway map based on the enhanced neural signals, identify an initiation site and a conduction pathway of peripheral nerve discharges, and output authentic discharge signals of the peripheral nerves.

10. The peripheral nerve signal acquisition system according to claim 9, wherein the orthogonal direction refers to a direction forming a 90° angle with a longitudinal axis of a forearm.

11. The peripheral nerve signal acquisition system according to claim 9, wherein a difference between a frequency of the mechanical perturbations and a natural mechanical resonance frequency of local tissues is less than a preset value.

12. The peripheral nerve signal acquisition system according to claim 9, wherein excitation of the perturbation unit is phase-offset from the electrical stimulation signal by half a cycle.

13. The peripheral nerve signal acquisition system according to claim 9, wherein the first signal and the second signal are routed to a general-purpose amplifier via a dual-channel differential mode.

14. The peripheral nerve signal acquisition system according to claim 9, wherein the interference signal set constitutes a two-dimensional matrix structure, and each element corresponds to a superimposed output of the first signal and the second signal under different delay combinations.

15. The peripheral nerve signal acquisition system according to claim 9, wherein the artifact template signal refers to a representative waveform curve generated from the electrophysiological signals acquired by the artifact detecting electrodes across a plurality of stimulation cycles through temporal alignment, average superposition, and filtering processing.

16. The peripheral nerve signal acquisition system according to claim 9, wherein the enhanced neural signals are acquired by performing artifact template fitting on respective channel signals within the interference signal set and suppressing the fitted components.
